(19) United States
(12) Patent Application Publication
(10) Pub. No.: US 2007/0086967 A1
(43) Pub. Date: Apr. 19, 2007

(54) Treatment of psoriasis with rosiglitazone

(76) Inventor: Brian MacDonald, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/596,169

(22) PCT Filed: Dec. 3, 2004
(86) PCT No.: PCT/US04/40441

§ 371 Date: Jun. 2, 2006

Related U.S. Application Data
(60) Provisional application No. 60/527,078, filed on Dec. 3, 2003.

(51) Int. Cl. A61K 8/72

(52) U.S. Cl. 424/70.11

(57) ABSTRACT

Disclosed is a method of treatment of psoriasis which comprises administering to a patient in need thereof by the oral route a pharmaceutical composition comprising rosiglitazone, or a pharmaceutically acceptable salt or solvate thereof, together with a pharmaceutically acceptable carrier, wherein the method comprises administering 2 to 8 mg rosiglitazone per day.

This invention relates to a novel therapeutic method, in particular to a method of treatment of psoriasis and to pharmaceutical compositions and their use in such method.

In the last decade or so a class of compounds known as thiazolidinediones (e.g. U.S. Pat. Nos. 5,089,514, 4,342,771, 4,367,234, 4,340,605, 5,306,726) have emerged as effective antidiabetic agents that enhance the insulin sensitivity of target tissues (skeletal muscle, liver, adipose) in animal models of non insulin dependent diabetes mellitus (“NIDDM”) and also reduce lipid and insulin levels in these animal models. The thiazolidinedione troglitazone was shown to have these same beneficial effects in human patients suffering from impaired glucose tolerance, a metabolic condition that precedes the development of NIDDM, as in patients suffering from NIDDM (J. J. Nolan et. al., N. Eng. J. Med. 1188-1193, 331 (1994)). While the mechanism of action is unclear, thiazolidinediones do not cause increases in insulin secretion or in the number or affinity of insulin receptor binding sites, suggesting that thiazolidinediones amplify post-receptor events in the insulin signaling cascade (J. R. Colca and D. R. Morton, “Antihyperglycemic thiazolidinediones: ciglitazone and its analogs,” in New Antidiabetic Drugs, C. J. Bailey and P. R. Flaft, eds., Smith-Gordon, New York, 255-261 (1990)).

Thiazolidinediones also induce the in vitro differentiation of preadipocyte cell lines into mature adipocytes (A. Hiragun, et. al. J. Cell. Physiol. 124-130, 134 (1988); R. F. Kleitzen, et. al., Mol. Pharmacol. 393-398, 41 (1992)). Treatment of pre-adipocyte cell lines with the thiazolidinedione pioglitazone results in increased expression of the adipocyte-specific genes aP2 and adipsin as well as the glucose transporter proteins GLUT-1 and GLUT-4, which suggests that the hypoglycaemic effects of thiazolidinediones seen in vivo may be mediated through adipose tissue.

More recently, an orphan member of the steroid/thyroid/retinoid receptor superfamily of ligand-activated transcription factors termed Peroxisome Proliferator-Activated Receptor gamma (PPAR-gamma) has been discovered. PPAR-gamma is one of a subfamily of closely related PPARs encoded by independent genes (C. Dreyer, et. al., Cell 879-887, 68 (1992); A. Schmidt, et. al., Mol. Endocrinol. 1634-1641, 6, (1992); Y. Zhu, et. al., J. Biol. Chem. 26817-26820, 268 (1993); S. A. Kliewer et. al., Proc. Nat. Acad. Sci. USA 7355-7359, 91, (1994)). Three mammalian PPARs have been isolated and termed PPAR-alpha, PPAR-gamma, and NUC-1, or PPARδ. These PPARs regulate expression of target genes by binding to DNA sequence elements, termed PPAR response elements (PPRE). To date, PPRE's have been identified in the enhancers of a number of genes encoding proteins that regulate lipid metabolism suggesting that PPARs play a pivotal role in the adipogenic signaling cascade and lipid homeostasis (H. Keller and W. Wahli, Trends Endocrin. Met 291-296, 4 (1993)). Thiazolidinediones are now known to be potent and selective activators of PPAR-gamma and bind directly to the PPAR-gamma receptor (J. M. Lehmann et. al., J. Biol. Chem. 12953-12956, 270 (1995)), providing evidence that PPAR-gamma is a possible target for the therapeutic actions of the thlazolidinediones. Indeed, since PPAR-gamma was identified as a key molecular target for thiazolidinediones, this nuclear transcription factor has been identified in a large number of human cell types, and thiazolidinediones have been claimed to have a broad spectrum of potential clinical utilities, for example in certain forms of cancer (e.g. G. D. Demetri et al., Proc. Natl. Acad. Sci. USA 3951-3956, 96 (1999)), multiple sclerosis (e.g. M. Niino et al., Neuroimmunology 40-48, 116 (2001)), Alzheimer's Disease (e.g. G. S. Watson and S. Craft, CNS Drugs 27-45, 17 (2003)), ulcerative colitis. (e.g. J. D. Lewis et al, Am. J. Gastroenterology 3323-3328, 96 (2001)), asthma (Y. Hashimoto and K. Nakahara, Diabetes Care 401, 25 (2002)) and vascular disease (e.g. J. Minamikawa et al, J. Clin. Endoctinol. Metab. 1818-1820, 83 (1998)). Many potential disease targets for thiazolidinediones have an inflammatory component, and it is possible that it is the muti-faceted anti-inflammatory effects of these drugs which will prove to be of critical therapeutic importance. In this respect, it is now known that thiazolidinediones can modulate the functions of white blood blood cells (e.g. R. Garg et al, Hypertension 430-435, 36 (2000); N. Marx et al, Circ. Res. 703-710, 90 (2002)) as well as reduce their number in the circulation (S. M. Haffner et al, Circulation 679-684, 106 (2002)).

U.S. Pat. No. 5,002,953 describes a class of thiazolidinedione derivatives for use as insulin sensitisers in the treatment of Type II diabetes mellitus. These compounds have anti-hyperglycaemic activity. One preferred compound described therein is known by the chemical name 5-[4-[2-(N-methyl-N-(2-pyridyl)amino)ethoxy]benzyl]thiazolidine-2,4-dione and has been given the generic name rosiglitazone. Salts of this compound including the maleate salt are described in WO94/05659. Certain pharmaceutical compositions are described in WO98/55122.

U.S. Pat. No. 5,594,015 (Kurtz et al) describes the use of certain thiazolidinedione derivatives including pioglitazone and ciglitazone for the treatment of psoriasis through a mechanism involving inhibition of proliferation of keratinocytes. This patent describes a range of presentations by which the drug substance may be administered to the patient, including, out of preference, by applying a cream or oil of around 1-2% strength directly to the psoriatic lesion, or else by administering the medication orally. Oral dosages are suggested to be in the range of 100-600 mg twice per day, eg 100-200 mg of compound twice per day. U.S. Pat. No. 6,403,656 (Rivier et al) reports the observation that the level of expression of PPAR gamma in psoriatic lesions is reduced relative to the healthy state. This patent describes the use of PPAR gamma agonists including rosiglitazone in the treatment of abnormalities of differentiation in epidermal cells, more particularly in the treatment of psoriasis, atopic dermatitis and eczema, acne, light induced keratosis and skin cancers. The compounds are indicated for enteral, parenteral and topical administration, generally at a daily dose of about 0.001-100 mg/kg of body weight, taken in 1 to 3 dosage intakes. Both Kurtz and Rivier performed their work on cultured keratinocytes.

Psoriasis is a debilitating autoimmune, dermatological, disease that affects about 1-3% of the population worldwide and 2.6% of the US population [National Psoriasis Foundation, 2002]. Plaque psoriasis, the most common form of the disease, is characterized by red skin covered with silvery scales. Histologically the picture is one of disordered differentiation and hyperproliferation of keratinocytes within the psoriatic plaque with inflammatory cell infiltrates [Ortonne J P, Brit Journal Dermatol (1999)140 (suppl 54) 1-7]. The psoriatic skin lesions are inflammatory, red, sharply delimited plaques of various shapes with characteristics silvery lustrous scaling. The erythema, skin thickening and scaling may cover an area of up to and sometimes exceeding 50% of the body surface. It is uncomfortable, disfiguring, and not satisfactorily treated by currently available medications.

As used herein “psoriasis” includes psoriasis and the symptoms of psoriasis including erythema, skin thickening/elevation and scaling.

The present inventors for the first time have demonstrated that rosiglitazone is very effective at treating psoriasis in humans, especially moderate to very severe psoriasis, when administered by the oral route at a dose of 2 to 8 mg/day.

Thus, according to the invention, there is provided a method of treatment of psoriasis which comprises administering to a patient in need thereof by the oral route a pharmaceutical composition comprising rosiglitazone, or a pharmaceutically acceptable salt or solvate thereof, together with a pharmaceutically acceptable carrier, wherein the method comprises administering 2 to 8 mg rosiglitazone per day.

According to a first embodiment of the invention the method comprises administering 2 mg rosiglitazone per day.

According to a second embodiment of the invention the method comprises administering 4 mg rosiglitazone per day.

According to a third embodiment of the invention the method comprises administering 8 mg rosiglitazone per day.

The method is suitable for psoriasis patients who also suffer from diabetes eg patients suffering from non insulin dependent diabetes mellitus (NIDDM). It is also suitable for psoriasis patients not suffering from diabetes eg NIDDM.

Preferably the method comprises administering the rosiglitazone once per day in a single dose or sequentially in two or more divided doses.

Preferably the rosiglitazone is administered in a unit dose eg 2, 3, 4, 5, 6, 7 or 8 mg especially 2 mg or alternatively 4 mg or alternatively 8 mg. Less preferably it may be administered in two or more divided doses eg 2 doses of 1 mg or 2 doses of 2 mg or 2 doses of 4 mg.

As can be seen from the Examples, statistically significant improvements have resulted from administering rosiglitazone for a continuous period of 12 weeks or more, more preferably 18 weeks or more. Rosiglitazone is expected to be most useful in long term maintenance therapy. This is all the more surprising since our investigations showed that rosiglitazone has a rather slow onset of action and that according to most measures onset of action occurs after around 2 weeks with onset of activity at around 6-8 weeks. Noticeably beneficial effects result after around 12-18 weeks of therapy. Thus, according to a preferred manner of performing the invention, the method comprises continuing treatment with rosiglitazone for 12 weeks or more, more preferably for 18 weeks or more. Treatment may be continued for 52 weeks or more.

It may be desired to administer rosiglitazone in combination with another substance considered to be effective in treating psoriasis eg:
biologics such as alefacept, etanercept, efalizumab, infliximab;
steroids especially Class 4 or Class 5 steroids such as hydrocortisone (eg 1% hydrocortisone cream);
cyclosporin or similar macrolide agent;
retinoids.

In a particularly preferred method of performing the invention a medication with fast onset of activity such as steroid therapy eg hydrocortisone may be employed to reduce symptoms during the initial period of onset of activity of rosiglitazone. “Fast” means fast relative to that of rosiglitazone, ideally with onset of activity within a 1 week, especially 1-2 days. For example such rescue medication may be used during the first 18 weeks of therapy eg during the first 12 weeks or the first 8 weeks of therapy on rosiglitazone.

Thus another aspect of the invention includes a method of treatment according to other aspects of the invention which also comprises administering another medicament effective in the treatment of psoriasis with fast onset of activity. The another medicament effective in the treatment of psoriasis with fast onset of activity is preferably administered until the rosiglitazone becomes effective (for example for a period of up to 18 weeks, eg up to 12 weeks, or up to 8 weeks of therapy of rosiglitazone). Preferably the therapy with the another medicament is discontinued after the initial period such that ongoing maintenance therapy is provided by rosiglitazone.

Preferably the another medicament is hydrocortisone eg 1% hydrocortisone cream.

Rosiglitazone may be employed as the free base however it is preferably employed as a pharmaceutically acceptable salt. The preferred salt is the maleate salt. Other possible salts include the hydrochloride salt.

Rosiglitazone and salts thereof may form solvates eg hydrates the use of which is embraced by the invention.

Severity of psoriasis can be diagnosed by one of a number of recognised scoring systems.

The Lattice System Global Psoriasis Score (LS-GPS) developed by Charles Ellis, M. D. is a physician's global assessment tool that provides an entire body assessment that combines total body surface area (BSA) involvement with average plaque qualities. The resulting LS-GPS is one of eight discrete values ranging from “Clear” to “Very Severe”.

Clear
Almost Clear
Mild
Mild to Moderate
Moderate
Moderate to Severe
Severe
Very Severe

According to the LS-GPS score the definitions are:
Clear
0% Body Surface Area (BSA) and no elevation, erythema or scale.
   Almost Clear:
1-3% BSA, mild elevation, erythema or scale; or
4-9% BSA, mild erythema or mild scale
   Mild:
1-3% BSA, moderate or marked elevation, erythema or scale; or
4-9% BSA, mild elevation or moderate erythema or moderate scale; or
10-20% BSA, mild erythema or mild/moderate scale
   Mild to Moderate:
4-9% BSA, moderate elevation or marked scale; or
10-20% BSA, moderate erythema; or
21-29% BSA, mild erythema or mild/moderate scale
   Moderate:
4-9% BSA, marked elevation or marked erythema; or
10-20% BSA, mild elevation; or
30-50% BSA, mild erythema or mild scale
   Moderate to Severe:
10-20% BSA, moderate elevation or marked scale; or
21-29% mild elevation or moderate erythema; or
51+% BSA, mild erythema or mild scale
   Severe:
10-20% BSA, marked elevation or marked erythema; or
21-29% BSA, moderate/marked elevation or marked erythema or marked scale; or
30-50% BSA, mild elevation or moderate erythema or moderate scale
   Very Severe:
30-50% BSA, moderate/marked elevation or marked erythema or marked scale; or
51+% BSA, any elevation or moderate/marked erythema or moderate/marked scale.

The Physicial's Global Assessment (PGA) is a 7 point scale used to measure the severity of disease at the time of the physician's evaluation. The PGS provides further assessment of disease activity and is relevant to clinical practice because many physician's rate disease activity on a scale ranging from “severe” to “clear”. The 7 point scale is:

Severe: very marked plaque elevation, scaling and/or erythema

Moderate to severe: marked plaque elevation, scaling and/or erythema

Moderate: moderate plaque elevation, scaling and/or erythema

Mild to moderate: mild plaque elevation, with moderate erythema and/or scale

Mild: mild plaque elevation, scale and/or erythema

Almost clear: slight elevation, scale and/or erythema

Clear: not signs of psoriasis (post inflammatory hypopigmentation or hyperpigmentation could be present).

The Psoriasis Area Severity Index (PASI) score was developed by Fredeniksson and Petersson in 1978 ( Frederiksson T and Petersson U. Severe psoriasis-oral therapy with a new retinoid. Dermatologica. 1978;157:238-44). According to our preferred method of determining the PASI score,four main body areas are assessed: the head (h), the upper extremities (u) the trunk (t), and lower extremities (l) corresponding to 10; 20, 30 and 40% of the total body surface area, respectively. The buttocks are counted as part of the legs, the axilla and groin count as part of the trunk and the neck as part of the head. The area of psoriatic involvement of these four main areas (Aₕ, Aₜ, Aᵤ, Aₗ) is given a numeric score for the percent involved by psoriasis: 1=1-9%; 2=10-29%; 3=30-49%; 4=50-69%; 5=70-89% and 6=90-100%. In order to evaluate the severity of the psoriatic lesions three target symptoms, erythema (E), thickness/indurabon (I), and desquamation/scaling (D) will be assessed according to a scale of 0-4, where 4 represents the severest possible involvement. In the case of i.e., erythema, 0=No symptoms; 1=slight erythema, 2=moderate erythema; 3=marked erythema, and 4=very marked erythema. The PASI score (0-72) is then calculated, from the following formula:

| | | Upper Ext. | |
|---|---|---|---|
| Head (H) | Trunk (T) | (U) | Lower Ext. (L) |
| 0.1 (E_{H} + I_{H} + D_{H}) | 0.3 (E_{T} + I_{T} + D_{T}) | 0.2 (E_{U} + I_{U} + D_{U}) | 0.4 (E_{L} + I_{L} + D_{L}) |
| A_{H} | A_{T} | A_{U} | A_{L} |

The PASI score varies in steps of 0.1 units from 0.0 to 72.0. Higher PASI scores indicate higher degrees of severity. The last mentioned score represents complete erythema of the severest possible degree, while 0.0 means no psoriatic lesions at all. Generally severe psoriasis is defined by a PASI score of 20 or more.

Preferably severity is determined by the LS-GPS scoring method.

The oral treatment with rosiglitazone is aimed primarily at the treatment of mild to very severe patients however we expect it to be particularly efficacious in the treatment of moderate to very severe, especially severe and very severe psoriasis.

According to one particular aspect of the invention there is provided a method of treatment of psoriasis in a patient which comprises
(a) diagnosing psoriasis in a patient; and
(b) administering to said patient by the oral route a pharmaceutical composition comprising rosiglitazone, or a pharmaceutically acceptable salt or solvate thereof, together with a pharmaceutically acceptable carrier, in an amount of 2 to 8 mg rosiglitazone per day.

More particularly, there is provided a method of treatment of moderate to very severe psoriasis in a patient which comprises
(a) diagnosing moderate to very severe psoriasis in a patient
(b) administering to said patient by the oral route a pharmaceutical composition comprising rosiglitazone, or a pharmaceutically acceptable salt or solvate thereof, together with a pharmaceutically acceptable carrier, in an amount of 2 to 8 mg rosiglitazone per day.

In step (b), the rosiglitazone is preferably administered once per day of treatment in a single dose or sequentially in two or more divided doses.

In step (b) rosiglitazone is preferably administered to a patient for a continuous period of 12 weeks or more, especially for a continuous period of 18 weeks or more.

A further aspects of the invention includes a kit for the treatment of psoriasis comprising:
(a) pharmaceutical composition comprising 2 to 8 mg rosiglitazone optionally in the form of a pharmaceutically acceptable salt or soivate thereof together with a pharmaceutically acceptable carrier in a single dose or in two or more divided doses per day of treatment; and
(b) instructions directing the oral administration by a patient suffering from psoriasis of 2 to 8 mg rosiglitazone optionally in the form of a pharmaceutically acceptable salt or solvate thereof together with a pharmaceutically acceptable carrier per day of treatment.

The kit may also contain another medicament effective in the treatment of psoriasis with fast onset of activity.

Preferably such a kit includes instructions to the patient to administer the another medicament effective in the treatment of psoriasis with fast onset of activity until the rosiglitazone becomes effective.

Preferably the another medicament is hydrocortisone.

We also provide use of rosiglitazone, or a pharmaceutically acceptable salt or solvate thereof, in the manufacture of a medicament for the oral treatment of psoriasis wherein said rosiglitazone, or a pharmaceutically acceptable salt or solvate thereof, is used in a pharmaceutical composition together with a pharmaceutically acceptable carrier, in an amount of 2 to 8 mg rosiglitazone per day of treatment.

We also provide use of rosiglitazone, or a pharmaceutically acceptable salt or solvate thereof, in the oral treatment of psoriasis wherein said rosiglitazone, or a pharmaceutically acceptable salt or solvate thereof, is used in a pharmaceutical composition together with a pharmaceutically acceptable carrier, in an amount of 2 to 8 mg rosiglitazone per day of treatment. We also provide use of rosiglitazone in combination with other active agents for example medicaments having fast onset of action as described above, especially for the initial period of therapy.

We also provide rosiglitazone, or a pharmaceutically acceptable salt thereof, for use in the treatment of psoriasis by the oral route in a pharmaceutical composition together with a carrier at a dosage of 2 to 8 mg rosiglitazone per day.

Rosiglitazone may exist in one of several tautomeric forms, all of which are included within the ambit of the invention. Rosiglitazone contains a chiral carbon atom and therefore can exist in two stereoisomeric forms. All forms whether as individual isomers or a mixture thereof (eg the racemate) are included within the ambit of the invention, although the racemate is preferred.

As used herein the term concentrate with respect to rosiglitazone means a proportionate amount of rosiglitazone greater than that present in an administerable composition.

For the avoidance of doubt, when reference is made herein to scalar amounts, including mg amounts and % weight amounts, of rosiglitazone as a pharmaceutically acceptable salt, or a solvate thereof, the scalar amount referred to is made in respect of rosiglitazone per se: For example 2 mg of rosiglitazone in the form of the maleate salt is that amount of maleate salt which contains 2 mg of rosiglitazone.

A process for preparing a pharmaceutical composition comprising 2 to 8 mg of rosiglitazone optionally in the form of a pharmaceutically acceptable salt or solvate thereof, and a pharmaceutically acceptable carrier therefor, comprises admixing 2 to 8 mg of rosiglitazone optionally in the form of a pharmaceutically acceptable salt or solvate thereof and the pharmaceutically acceptable carrier and optionally thereafter formulating the composition produced into an administerable form.

A particular process for preparing a pharmaceutical composition of rosiglitazone optionally in the form of a pharmaceutically acceptable salt or solvate thereof and a pharmaceutically acceptable carrier, comprises:
(i) preparing a pre-administration composition comprising rosiglitazone optionally in the form of a pharmaceutically acceptable salt or solvate thereof and a first pharmaceutically acceptable carrier;
(ii) admixing the pre-administration composition with a second pharmaceutically acceptable carrier to provide the required composition of rosiglitazone and optionally thereafter formulating the composition produced into an administerable form.

A preferred administerable form of the pharmaceutical composition of rosiglitazone is a unit dose composition.

Suitable unit doses comprise up to 8 mg, such as 2 to 8 mg, of rosiglitazone optionally in the form of a pharmaceutically acceptable salt or solvate thereof.

A suitable pharmaceutically acceptable, pre-administration composition is a concentrate, preferably a granular concentrate, of rosiglitazone optionally in the form of a pharmaceutically acceptable salt or solvate thereof. The granular concentrate is particularly well adapted to be diluted to provide a composition for administration, preferably a tablet.

Suitably, the pre-administration composition contains up to 50% by weight, for example an amount in the range of from 2 to 50% by weight, of rosiglitazone optionally in the form of a pharmaceutically acceptable salt or solvate thereof.

Favourably, the pre-administration composition contains an amount of rosiglitazone optionally in the form of a pharmaceutically acceptable salt or solvate thereof in the range of from 5 to 20% by weight, in particular 5%, 10% or 15% by weight, for example 10% by weight.

The above mentioned processes can provide pharmaceutical compositions of rosiglitazone in any conventionally orally administerable form.

The first pharmaceutically acceptable carrier can comprise any conventional pharmaceutically acceptable carrier comprising conventional pharmaceutically acceptable excipients, including those disclosed in the reference texts mentioned below. However, as it is not essential that the first pharmaceutically acceptable carrier is in an administerable form, then it need not contain excipients solely associated with administration. For example the first pharmaceutically acceptable carrier need not contain a lubricant.

The second pharmaceutically acceptable carrier includes any conventional pharmaceufically acceptable carrier comprising any conventional pharmaceutically acceptable excipient, including disintegrants, diluents and lubricants, including those disclosed in the reference texts mentioned below.

One particular pre-administration composition comprises rosiglitazone optionally in the form of a pharmaceutically acceptable salt or solvate thereof, a disintegrant, a binder and a diluent.

A suitable disintegrant is sodium starch glycollate.

A suitable binder is a methyl cellulose binder, such as hydroxypropyl methylcellulose 2910.

Suitable diluents include cellulose, for example a microcrystalline cellulose, and lactose monohydrate.

A suitable lubricant is magnesium stearate.

We have found that a particularly advantageous first composition contains rosiglitazone optionally in the form of a pharmaceutically acceptable salt or solvate thereof, sodium starch glycollate, hydroxypropyl methylcellulose 2910, microcrystalline cellulose and lactose monohydrate, especially when in a granular form. This granular form has been found to be particularly stable.

When the pre-administration composition contains about 10% by weight of rosiglitazone optionally in the form of a pharmaceutically acceptable salt or solvate thereof, it is readily dilutable to give unit dose compositions comprising in the range of between 2 to 8 mg, 2 to 4 mg and 4 to 8 mg rosiglitazone optionally in the form of a pharmaceutically acceptable salt or solvate thereof.

The preparation of the pre-administration composition is suitably carried using any conventional method appropriate to the nature of the said first composition, for example wet granulation methods provide the first composition in granular form.

Methods for formulating the compositions of the invention into administerable forms include conventional formulation methods as disclosed in the reference texts cited herein, including tabletting methods.

The orally administerable compositions may be in the form of tablets, capsules, powders, granules, lozenges, reconstitutable powders, or liquid preparations, such as oral solutions or suspensions.

In order to obtain consistency of administration it is preferred that a composition of the invention is in the form of a unit dose.

Unit dose presentation forms for oral administration may be tablets and capsules and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulphate.

Unless otherwise prescribed, compositions of the invention are preferably in unit dosage form in an amount appropriate for the relevant daily dosage, suitable unit dosages comprise 2, 3, 4, 5, 6, 7 or 8 mg of rosiglitazone optionally in the form of a pharmaceutically acceptable salt or solvate thereof. Alternatively, but less preferably, the daily dose is divided eg a dose of 1 mg may be given more than once.

The solid oral compositions may be prepared by conventional methods of blending, filling or tabletting. As required repeated blending operations may be used to distribute the active agent throughout those compositions employing large quantities of fillers. Such operations are of course conventional in the art. The tablets may be coated according to methods well known in normal pharmaceutical practice, in particular with an aqueous film coating.

Liquid compositions, for example oral liquid compositions, may be in the form of emulsions, syrups, or elixirs, or they may be in a dry product form to be reconstituted with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

Solid dosage forms are preferred eg tablets or capsules, especially tablets.

Unless otherwise specified the compositions of the invention may contain from 0.1% to 99% by weight, preferably from 10-60% by weight, of the active material, depending upon the method of administration.

The composition may, if desired, be in the form of a pack accompanied by written or printed instructions for use.

Compositions may be prepared and formulated according to conventional methods, such as those disclosed in standard reference texts, for example the British and US Pharmacopoeias, Remington's Pharmaceutical Sciences (Mack Publishing Co.), Martindale The Extra Pharmacopoeia (London, The Pharmaceutical Press) and Harry's Cosmeticology (Leonard Hill Books).

### BRIEF DESCRIPTION OF THE FIGURES

FIG. 1: graph showing percentage of completed patients who fell within the clear, almost clear or mild status for the 2, 4 and 8 mg once daily doses on the LS-GPS scale after 6, 12 and 18 weeks.

FIG. 2: graph showing percentage change in number of patients (all patients: last observation carried forward) moving from baseline on the PASI scale for the 2, 4 and 8 mg once daily doses.

FIGS. 3, 4, 5 and 6: photographs of the back of a patient receiving 2 mg rosiglitazone at baseline and after 12 weeks, 18 weeks and follow-up (week 20).

FIGS. 7, 8 and 9: photographs of the lower leg of a patient receiving 8 mg rosiglitazone at baseline and after 12 weeks and 18 weeks.

The following examples illustrate the invention but do not limit it in any way

### PREPARATIVE EXAMPLES FOR ROSIGLITAZONE

### Example 1

Concentrate Preparation

Approximately two thirds of the lactose monohydrate is passed through a suitable screen and blended with the milled maleate salt of rosiglitazone.

Sodium starch glycollate, hydoxypropyl methylcellulose, microcrystalline cellulose and the remaining lactose are passed through a suitable screen and added to the mixture. Blending is then continued. The resulting mixture is then wet granulated with purified water. The wet granules are then screened, dried on a fluid bed drier and the dried granules are passed through a further screen and finally homogenised.

% COMPOSITION OF GRANULAR CONCENTRATE

| Ingredient | Quantity (%) |
|---|---|
| Milled rosiglitazone as | 13.25 (pure maleate salt) |
| maleate salt | |
| Sodium Starch Glycollate | 5.00 |
| Hydoxypropyl | 5.00 |
| Methylcellulose 2910 | |
| Microcrystalline Cellulose | 20.0 |
| Lactose Monohydrate, | To 100 |
| regular grade | |
| Purified water | * |

* Removed during processing.

### Example 2

Formulation of the Concentrate into Tablets.

The granules from Example 1 are placed into a tumble blender. Approximately two thirds of the lactose is screened and added to the blender. The microcrystalline cellulose, sodium starch glycollate, magnesium stearate and remaining lactose are screened and added to the blender and the mixture blended together. The resulting mix is then compressed on a rotary tablet press to a target weight of 150 mg for the 1, 2 and 4 mg tablets and to a target weight of 300 mg for the 8 mg tablets.

The tablet cores are then transferred to a tablet coating machine, pre-warmed with warm air (approximately 65° C.) and film coated until the tablet weight has increased by 2.0% to 3.5%

| | Quantity (mg per Tablet) | | | |
|---|---|---|---|---|
| Tablet Strength | 1.0 mg | 2.0 mg | 4.0 mg | 8.0 mg |
| Active Ingredient: | | | | |
| Rosiglitazone maleate Concentrate | 10.00 | 20.00 | 40.00 | 80.00 |
| granules | | | | |
| Other Ingredients: | | | | |
| Sodium Starch Glycollate | 6.96 | 6.46 | 5.46 | 10.92 |
| Microcrystalline Cellulose | 27.85 | 25.85 | 21.85 | 43.70 |
| Lactose monohydrate | 104.44 | 96.94 | 81.94 | 163.88 |
| Magnesium Stearate | 0.75 | 0.75 | 0.75 | 1.50 |
| Total Weight of Tablet Core | 150.0 | 150.0 | 150.0 | 300.0 |
| Aqueous film coating material | 4.5 | 4.5 | 4.5 | 9.0 |
| Total Weight of Film Coated | 154.5 | 154.5 | 154.5 | 309.0 |
| Tablet | | | | |

### Biological Data on Rosiglitazone

### Example 3

Clinical Studies on Psoriasis Patients

A phase II, multi-center, randomized, double-blind, placebo-controlled study involving 186 subjects compared the safety and efficacy of three doses (2 mg, 4 mg, and 8 mg) of rosiglitazone maleate (RSG) to placebo for eighteen weeks with two weeks follow-up in the treatment of moderate to severe plaque psoriasis. This study was designed to provide an assessment of the risk to benefit profile of three dose levels of RSG in subjects with psoriasis. 186 patients were enrolled and 108 patients completed the study. All patients were in the moderate to very severe category, although of the patients starting the study only 11 were moderate and the rest were severe or very severe.

Efficacy measurements included the Lattice System Global Psoriasis Score (LS-GPS) and the Psoriasis Area and Severity Index (PASI).

At Week 12, the primary endpoint was the proportion of subjects in each treatment group, who achieved Clear/Almost Clear in the LS-GPS. At Week 12, one subject in each of the RSG groups achieved this status, compared with none in the placebo group. Secondary objectives, which evaluated PASI and additional aspects of LS-GPS results at Week 12, support promising indications of efficacy. The RSG 2 mg and 8 mg group demonstrated statistically significant results in the mean percent change in PASI at Week 12. Also, the proportion of subjects who achieved a 75% improvement according the PASI, proportion of subjects who achieved a 50% improvement according the PASI, and the LS-GPS summary all demonstrated improvements in the RSG 2 mg and 8 mg groups compared to placebo.

Continued improvement was observed after 18 weeks of treatment for the RSG groups compared with the placebo group. Specifically, at Week 18, six subjects achieved an improvement of Clear/Almost Clear, and these subjects were all on RSG. The distribution of the LS-GPS was statistically significant for the RSG 2 mg group. For the other LS-GPS endpoints, the 8 mg dose had comparable efficacy with the 2 mg dose, with the exception of the proportion of subjects who achieved Clear/Almost Clear/Mild according to the LS-GPS. For the proportion of subjects who achieved Clear/Almost Clear/Mild, the 8 mg group achieved statistical significance with the odds for successful treatment (defined as Clear/Almost Clear/Mild LS-GPS) estimated at 5 times greater than for the placebo group at Week 18. Given that all but 11 subjects started in the bottom three categories of Moderate-Severe, Severe and Very Severe, 25% of all subjects who took 8 mg RSG, moved to the top three categories (Clear/Almost Clear/Mild) versus placebo 6%, 2 mg RSG 10% and 4 mg RSG 9%. Additionally, the PASI mean percent change at Week 18 was statistically significant for the RSG 2 mg group. For the other PASI endpoints, the 8 mg dose had comparable efficacy with the 2 mg dose.

The results suggest that rosiglitazone maleate has a long onset of effect. Although the mean percent change in PASI for the 2 mg and 8 mg dose suggests onset-of-action as early as two weeks, other endpoints indicate that peak effect occurs later, at 18 weeks. In fact, peak efficacy may not have been observed as the study was only 18 weeks.

Improvements according to the mean percent change in the PASI total scores noted after 18 weeks of treatment were maintained in all treatment groups during the follow-up period.

The RSG 4 mg group results did not separate appreciably from placebo. However, the high dropout rate from this group (24/44) may have confounded the efficacy results of this dose group. This study had a high overall withdrawal rate at 48% and is thought to be due to the longer-than expected onset of effect of RSG. This high withdrawal rate was accentuated in the 4 mg group. Because the RSG 4 mg group had only 16 subjects who completed the study, clear conclusions regarding efficacy in this treatment group can not be made. However efficacy would have been expected based on the data in patients with 2 mg and 8 mg.

The safety profile was comparable across treatment groups. There were no unexpected safety findings in subjects after 18 weeks of oral, daily treatment of RSG doses.

The data is illustrated in the figures and in the following table:

| Efficacy measure | Placebo | 2 mg | 4 mg | 8 mg |
|---|---|---|---|---|
| Improvement to | | | | |
| clear/almost clear | | | | |
| LOCF | 0 | 2 (4%) | 1 (2%) | 3 (7%) |
| Observed | 0 | 2 (5%) | 1 (5%) | 3 (11%) |
| Improvement to | | | | |
| clear/almost | | | | |
| clear/mild | | | | |
| LOCF | 3 (6%) | 5 (10%) | 4 (9%) | 11 (25%) |
| Observed | 2 (8%) | 5 (13%) | 2 (10%) | 10 (37%) |
| Improvement by | | | | |
| 2 categories | | | | |
| LOCF | 6 (13%) | 12 (24%) | 6 (14%) | 11 (25%) |
| Observed | 4 (16%) | 11 (28%) | 4 (20%) | 10 (37%) |

The table shows the number of patients and the percentage reaching the efficacy measure after 18 weeks. “Observed” refers to data on patients who completed the trial. “LOCF” (last observation carried forward) includes data on patients who withdrew from the trial for some reason and includes their last observation in the data. The percentage is out of the total number of initial participants in the trial.

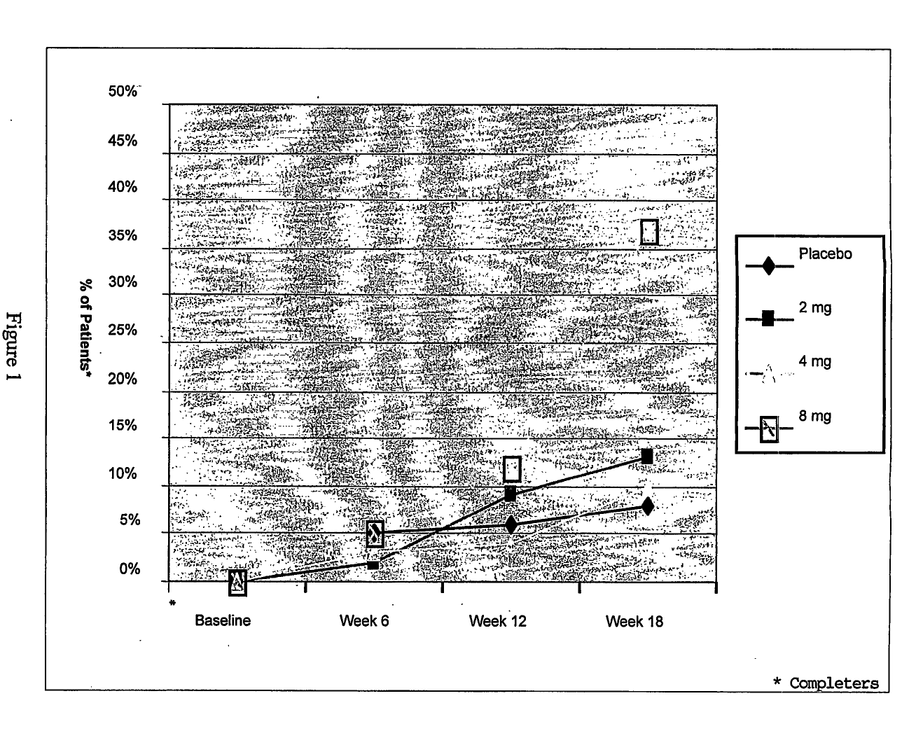

FIG. 1 shows particularly good LS-GPS results in the 8 mg group at 18 weeks.

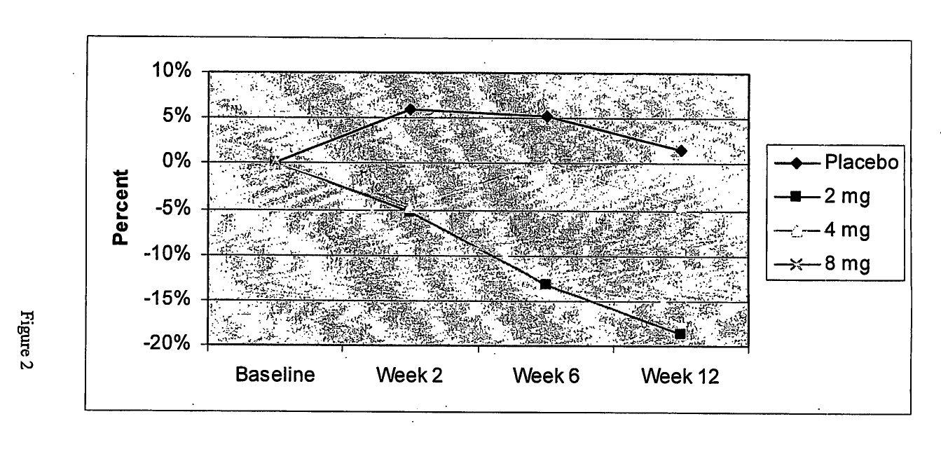

FIG. 2 shows particularly good PASI results in the 2 and 8 mg group at 12 weeks.

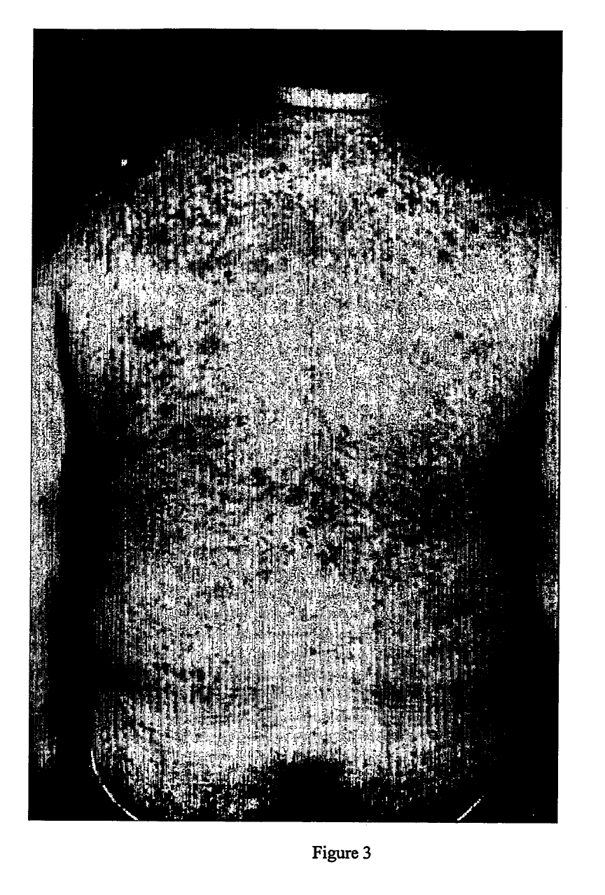

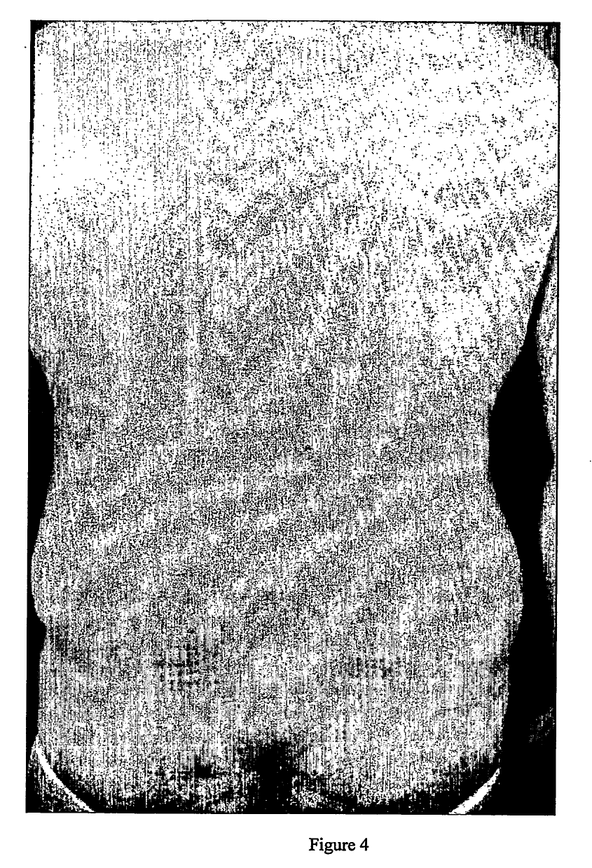

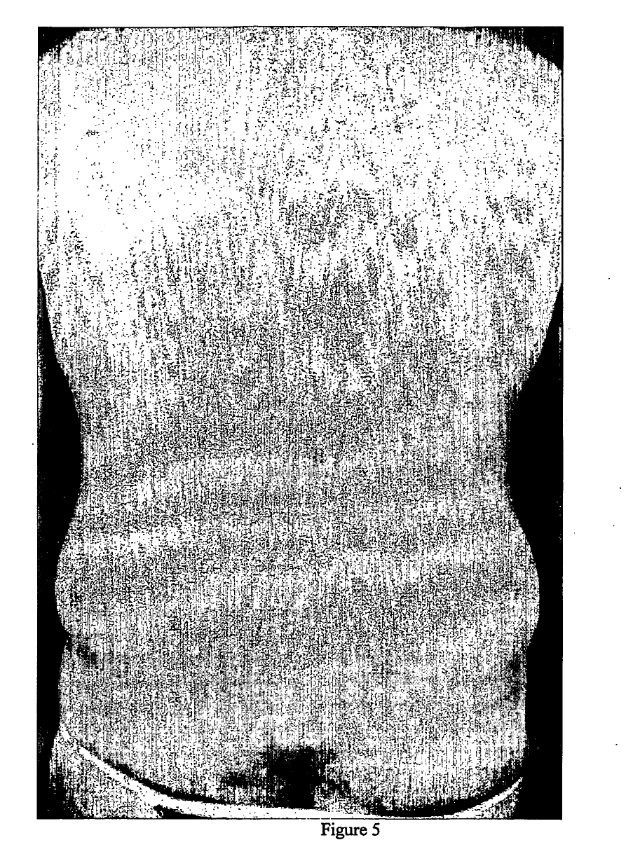

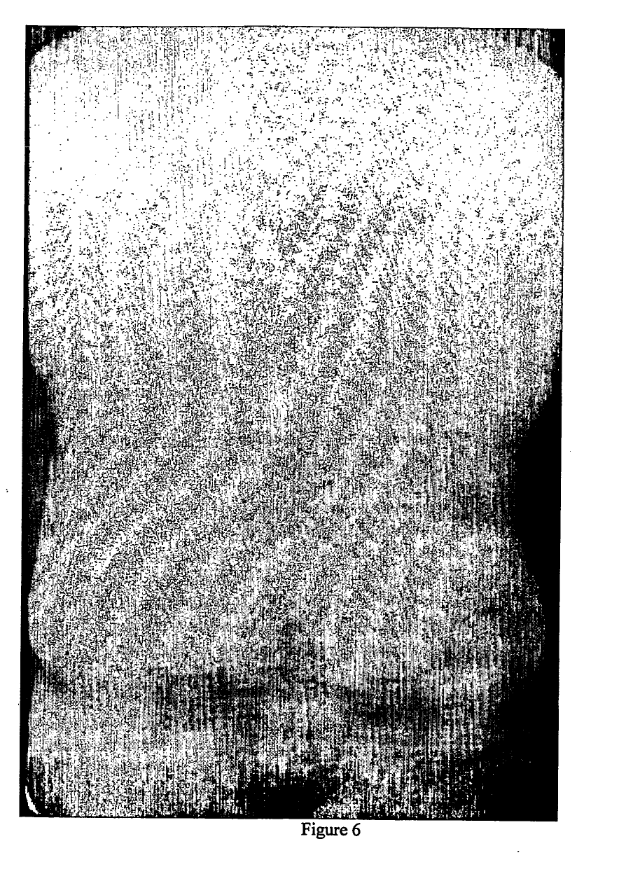

FIGS. 3-6 shows good efficacy in a patient on 2 mg RSG.

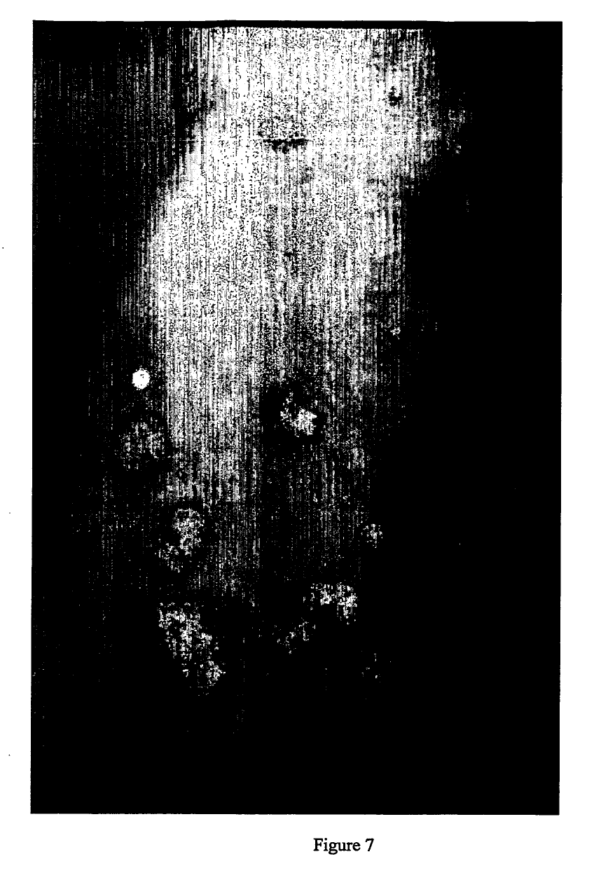

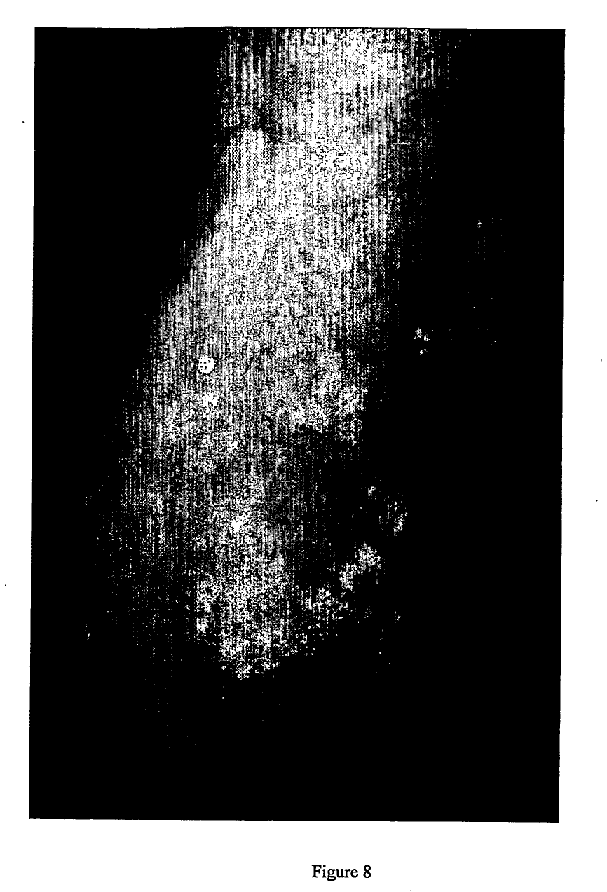

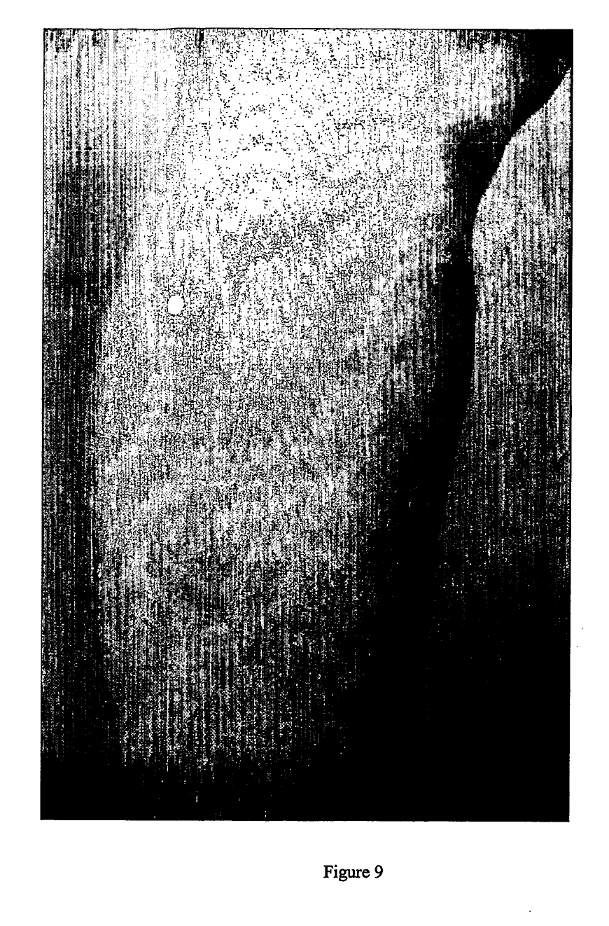

FIGS. 7-9 shows good efficacy in a patient on 8 mg RSG.

In Summary:

Adverse events were comparable with placebo and all doses were well tolerated.

Clinically meaningful effects were observed at Week 12 and Week 18 with both the 2 mg and 8 mg treatment groups. Longer term therapy may be required to maximize benefit.

Results of this study indicate that RSG shows considerable promise as a safe, oral therapeutic option for long term treatment and maintenance of plaque psoriasis.

Patents and patent applications mentioned above are hereinbefore incorporated by reference in their entirety.

1. A method of treatment of psoriasis which comprises administering to a patient in need thereof by the oral route a pharmaceutical composition comprising rosiglitazone, or a pharmaceutically acceptable salt or solvate thereof, together with a pharmaceutically acceptable carrier, wherein the method comprises administering 2 to 8 mg rosiglitazone per day.

2. A method according to claim 1 wherein the method comprises administering 2 mg rosiglitazone per day.

3. A method according to claim 1 wherein the method comprises administering 4 mg rosiglitazone per day.

4. A method according to claim 1 wherein the method comprises administering 8 mg rosiglitazone per day.

5. A method according to claim 1 wherein the method comprises administering the rosiglitazone once per day in a single dose or sequentially in two or more divided doses.

6. A method according to claim 1 wherein the patient is suffering from moderate to very severe psoriasis.

7. A method according to claim 1 wherein the treatment is continued for 12 weeks or more.

8. A method according to claim 7 wherein the treatment is continued for 18 weeks or more.

9. A method according to claim 1 wherein the rosiglitazone is employed as its maleate salt.

10. A method of treatment of psoriasis in a patient which comprises
(a) diagnosing psoriasis in a patient
(b) administering to said patient by the oral route a pharmaceutical composition comprising rosiglitazone, or a pharmaceutically acceptable salt or solvate thereof, together with a pharmaceutically acceptable carrier, in an amount of 2 to 8 mg rosiglitazone per day.

11. A method according to claim 10 which is a method of treatment of moderate to very severe psoriasis and step (a) comprises diagnosing moderate to very severe psoriasis in the patient.

12. A method according to claim 10 wherein in step (a) the patient is diagnosed with severe or very severe psoriasis.

13. A method according to claim 10 wherein in step (b) the rosiglitazone is administered once per day in a single dose or sequentially in two or more divided doses.

14. A method according to claim 10 wherein in step (b) rosiglitazone is administered to a patient for a continuous period of 12 weeks or more.

15. A method according to claim 14 wherein in step (b) rosiglitazone is administered to a patient for a continuous period of 18 weeks or more.

16. A method according to claim 1 which also comprises administering another medicament effective in the treatment of psoriasis with fast onset of activity.

17. A method according to claim 16 wherein the another medicament effective in the treatment of psoriasis with fast onset of activity is administered until the rosiglitazone becomes effective.

18. A method according to claim 16 wherein the another medicament is hydrocortisone.

19. A method according to claim 1 wherein the patient also suffers from non-insulin dependent diabetes mellitus.

20. A method according to claim 1 wherein the patient does not also suffers from non-insulin dependent diabetes mellitus.

21. A kit for the treatment of psoriasis comprising:
(a) A pharmaceutical composition comprising 2 to 8 mg rosiglitazone optionally in the form of a pharmaceutically acceptable salt or solvate thereof together with a pharmaceutically acceptable carrier in a single dose or in two or more divided doses per day of treatment; and
(b) instructions directing the oral administration by a patient suffering from psoriasis of 2 to 8 mg rosiglitazone optionally in the form of a pharmaceutically acceptable salt or solvate thereof together with a pharmaceutically acceptable carrier per day of treatment.

22. A kit according to claim 21 wherein the instructions direct the the oral administration by a patient suffering from psoriasis of 2 to 8 mg rosiglitazone optionally in the form of a pharmaceutically acceptable salt or solvate thereof together with a pharmaceutically acceptable carrier once per day of treatment in a single dose or sequentially in two or more divided doses.

23. A kit according to claim 21 wherein the rosiglitazone is employed as its maleate salt.

24. A kit according to claim 21 which also contains another medicament effective in the treatment of psoriasis with fast onset of activity.

25. A kit according to claim 24 including instructions to the patient to administer the another medicament effective in the treatment of psoriasis with fast onset of activity is administered until the rosiglitazone becomes effective.

26. A kit according to claims 24 wherein the medicament is hydrocortisone.

27. A kit according to claim 21 wherein the patient also suffers from non-insulin dependent diabetes mellitus.

28. A kit according to claim 21 wherein the patient does not also suffer from non-insulin dependent diabetes mellitus.

29-45. (canceled)
